(19) United States
(12) Patent Application Publication
(10) Pub. No.: US 2004/0267294 A1
(43) Pub. Date: Dec. 30, 2004

(54) Eye fixation apparatus

(76) Inventor: Brian R. Will, Battle Ground, WA (US)

Correspondence Address: K.M. RYLANDER TRIAL & PATENT ATTORNEY AT LAW PC, Suite 206, 1014 Franklin Street, Vancouver, WA, 98660, US

(21) Appl. No.: 10/608,408

(22) Filed: Jun. 27, 2003

(51) Int. Cl. A61B 17/00; A61F 9/00

(52) U.S. Cl. 606/166; 606/1

(57) ABSTRACT

An eye fixation apparatus including an eye fixation portion with an annular convex bottom, having criss-crossing channels, which goes upon the surface of an eyeball and encircles the cornea, a vacuum port in communication with the criss-crossing channels to exert vacuum pressure through the channels to pull the eyeball membrane to the bottom, and adjustment arms An eye fixation apparatus can further be provided with an X-translation guide member to adjustably moveable in the X-translation direction in relation to the eye fixation portion, a Y-translation guide member adjustably moveable in the Y-translation direction in relation to the eye fixation portion, and a docking screw.

### FIELD OF THE INVENTION

The present invention relates to devices and methods for fixating eyes for ophthalmic surgery, and more particularly to eye fixation devices and methods using vacuum pressure for fixation for guiding a surgical tool or laser.

### BACKGROUND

Ophthalmic surgery typically requires fixating the eye so that the eye is stabilized from movement during surgery. Articles and methods for fixating the eye are well known in the art. There are known annuli for fixating the eye which have soft, spreadable rings which spread to conform to the shape of the eye, and then vacuum pressure, usually in the form of suction, is applied. Known articles and methods typically involve a annulus which encircles the cornea, and which has an open bottom, through which vacuum pressure is exerted, sucking the eye globe conjunctiva and attached sclera into the bottom of the annulus, thus stabilizing the annulus on the eye. Among the know art are annuli which are hard and which have an exposed channel which contacts the eye. Vacuum pressure runs through that exposed channel, sucking the eye globe conjunctiva and attached sclera into the channel. This deforms the eye globe conjunctiva and attached sclera into the shape of the channel ring. Thus, if the fixation needs to be adjusted, a deformation ring still exists, which the annulus naturally settles back down upon again. This interferes with the ability to adjust the fixation device in the event of improper alignment.

The following represents a list of known related art:

| Reference: | Issued to: | Date of Issue: |
|---|---|---|
| U.S. Pat. No. 6,342,053 B1 | Berry | Jan. 29, 2002 |
| U.S. Pat. No. 6,338,710 B1 | Takahashi et al | Jan. 15, 2002 |
| U.S. Pat. No. 6,254,595 B1 | Juhasz et al | Jul. 3, 2001 |
| U.S. Pat. No. 6,231,585 B1 | Takahasi et al | May 15, 2001 |
| U.S. Pat. No. 6,099,541 | Klopotek | Aug. 8, 2000 |
| U.S. Pat. No. 6,071,295 | Takahashi | Jun. 6, 2000 |
| U.S. Pat. No. 6,030,398 | Klopotek | Feb. 29, 2000 |
| U.S. Pat. No. 5,820,624 | Yavitz | Oct. 13, 1998 |
| U.S. Pat. No. 5,817,115 | Nigam | Oct. 6, 1998 |
| U.S. Pat. No. 5,807,380 | Dishler | Sep. 15, 1998 |
| U.S. Pat. No. 5,695,492 | Brown | Dec. 9, 1997 |
| U.S. Pat. No. 5,649,922 | Yavitz | Jul. 22, 1997 |
| U.S. Pat. No. 5,601,548 | Smith et al | Feb. 11, 1997 |
| U.S. Pat. No. 5,586,980 | Kremer et al | Dec. 24, 1996 |
| U.S. Pat. No. 5,582,608 | Brown | Dec. 10, 1996 |
| U.S. Pat. No. 5,569,280 | Kamerling | Oct. 29, 1996 |
| U.S. Pat. No. 5,556,417 | Sher | Sep. 17, 1996 |
| U.S. Pat. No. 5,556,406 | Gordon et al | Sep. 17, 1996 |
| U.S. Pat. No. 5,336,215 | Hsueh et al | Aug. 9, 1994 |
| U.S. Pat. No. 5,171,254 | Sher | Dec. 15, 1992 |
| U.S. Pat. No. 5,108,412 | Krumeich et al | Apr. 28, 1992 |
| U.S. Pat. No. 5,092,863 | Schanzlin | Mar. 3, 1992 |
| U.S. Pat. No. 5,009,660 | Clapham | Apr. 23, 1991 |
| U.S. Pat. No. 4,905,711 | Bennett et al | Mar. 6, 1990 |
| U.S. Pat. No. 4,718,418 | L'Esperance, Jr. | Jan. 12, 1988 |
| U.S. Pat. No. 4,688,570 | Kramer et al | Aug. 25, 1987 |
| U.S. Pat. No. 4,173,980 | Curtin | Nov. 13, 1979 |
| U.S. Pat. No. 3,074,407 | T. E. Moon et al | Jan. 22, 1963 |
| EP 0372127A1 | L Esperance | Jun. 13, 1990 |
| U.S. Des. Patent 364,681 | Livernois | Nov. 28, 1995 |

The teachings of each of the above-listed citations (which does not itself incorporate essential material by reference) are herein incorporated by reference. None of the above inventions and patents, taken either singularly or in combination, is seen to describe the instant invention as claimed.

Thus, while the foregoing body of art indicates it to be well known to have an eye fixation devices for ophthalmic procedures, the art described above does not teach or suggest an eye fixation apparatus which has the following combination of desirable features: (1) functions without the need for a lid speculum; (a) low profile fits comfortably under the lids; (b) can more easily be used in patients with “tight lids” which are common to some races; (2) multi-point fixation as opposed to two point fixation to the surface of the eye; (3) markedly decreased deformation of the eye; (4) significantly reduces elevation of intraocular pressure: (a) safer; (b) more comfortable; (c) improves accuracy in femtosecond procedures: (5) decreases trauma to ocular surface and conjunctiva; (6) easier to reposition on globe of the eye if initial ring position is not suitable: (7) X and Y adjustment allows for superior centration properties; and (8) fixation screw is superior to pincer type fixation: (a) smoother docking possible; and (b) less manual dexterity required.

### SUMMARY AND ADVANTAGES

An eye fixation apparatus of the present invention includes an eye fixation portion with a contact portion, having criss-crossing channels, which goes upon the surface of an eyeball and encircles the cornea, a vacuum port in communication with the criss-crossing channels to exert vacuum pressure through the channels to pull the eyeball to the bottom, and adjustment arms An eye fixation apparatus can further be provided with an X-translation guide member adjustably moveable in the X-translation direction in relation to the eye fixation portion, a docking screw, and a Y-translation guide member adjustably moveable in the Y-translation direction in relation to the eye fixation portion.

The eye fixation apparatus of the present invention presents numerous advantages, including: (1) functions without the need for a lid speculum; (a) low profile fits comfortably under the lids; (b) can more easily be used in patients with “tight lids” which are common to some races; (2) multi-point fixation as opposed to two point fixation to the surface of the eye; (3) markedly decreased deformation of the eye; (4) significantly reduces elevation of intraocular pressure: (a) safer; (b) more comfortable; (c) improves accuracy in femtosecond procedures: (5) decreases trauma to ocular surface and conjunctiva; (6) easier to reposition on globe of the eye if initial ring position is not suitable: (7) X and Y adjustment allows for superior centration properties; and (8) fixation screw is superior to pincer type fixation: (a) smoother docking possible; and (b) less manual dexterity required.

Additional advantages of the invention will be set forth in part in the description which follows, and in part will be obvious from the description, or may be learned by practice of the invention. The advantages of the invention may be realized and attained by means of the instrumentalities and combinations particularly pointed out in the appended claims. Further benefits and advantages of the embodiments of the invention will become apparent from consideration of the following detailed description given with reference to the accompanying drawings, which specify and show preferred embodiments of the present invention.

### BRIEF DESCRIPTION OF THE DRAWINGS

FIG. 1 shows a perspective view of an embodiment of the present invention

FIG. 2 displays a top down view illustrating the X-Y adjustment capability of an embodiment of the present invention.

FIG. 3 shows the bottom view of the eye fixation portion of the present invention.

FIG. 4 shows a side view of the eye fixation portion of the present invention.

FIG. 5 shows an exploded view of an embodiment of the present invention.

FIG. 6 shows a detail of FIG. 5.

### DETAILED DESCRIPTION

Before beginning a detailed description of the subject invention, mention of the following is in order. When appropriate, like reference materials and characters are used to designate identical, corresponding, or similar components in differing figure drawings. The figure drawings associated with this disclosure typically are not drawn with dimensional accuracy to scale, i.e., such drawings have been drafted with a focus on clarity of viewing and understanding rather than dimensional accuracy.

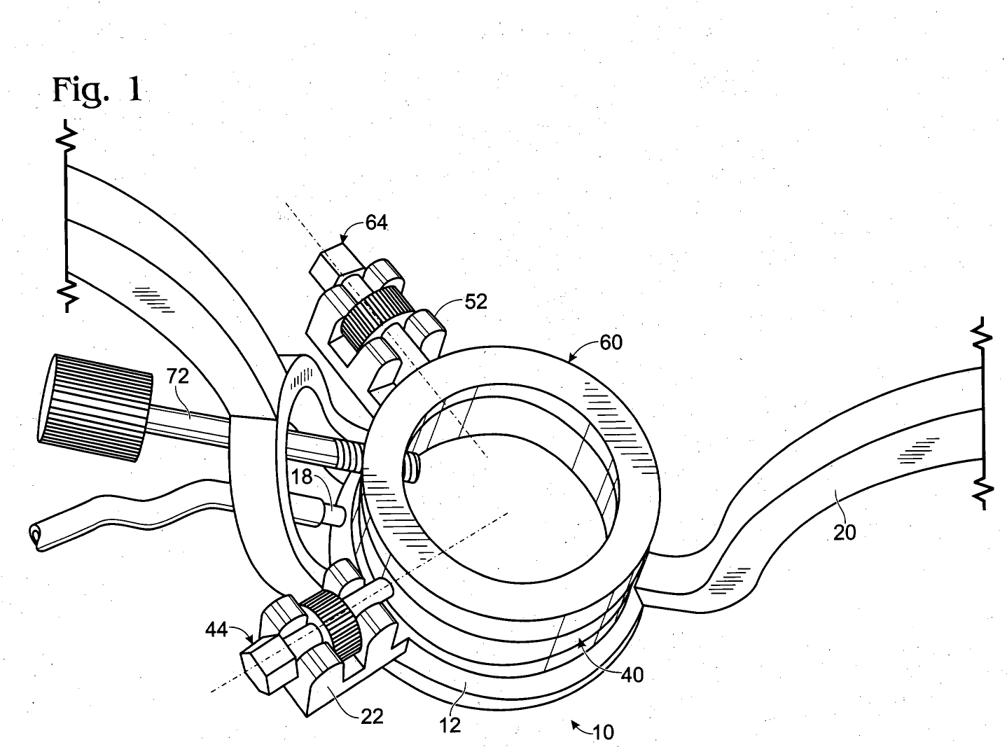

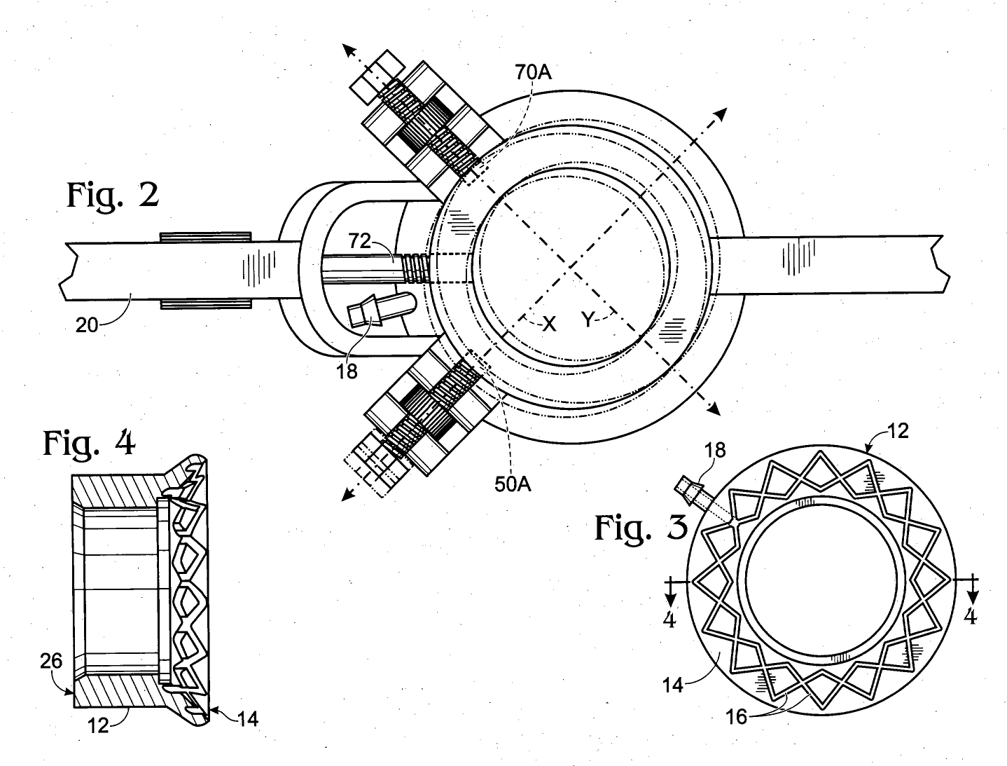

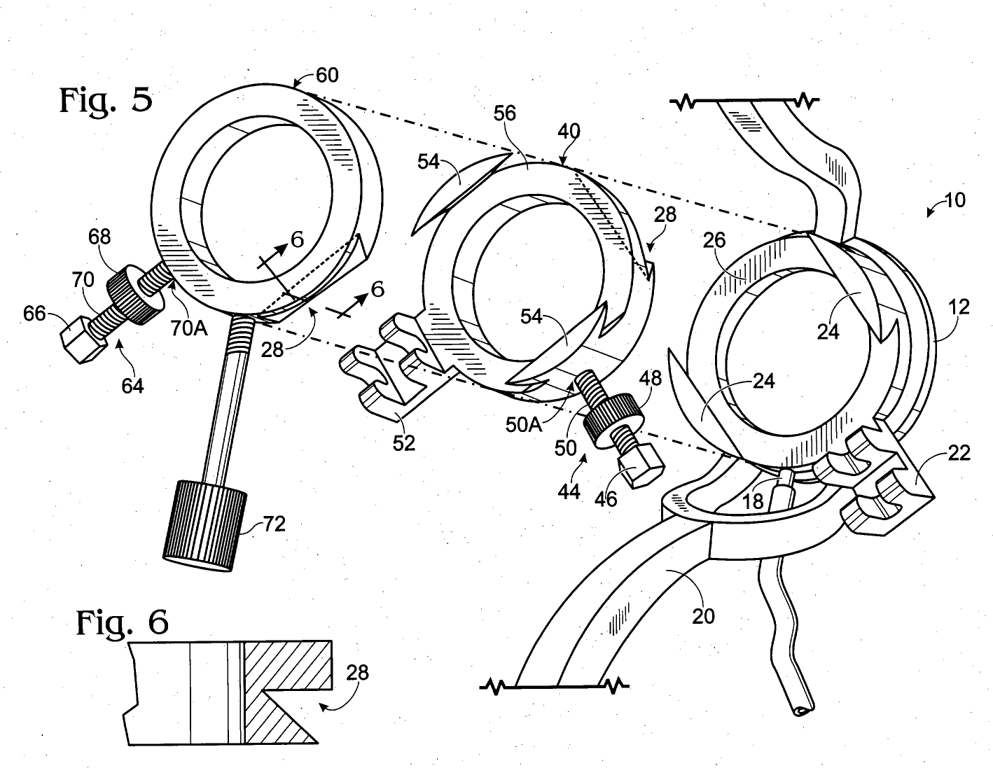

As shown in FIGS. 1 and 5, an eye fixation apparatus 10 is provided. Eye fixation apparatus 10 includes an eye fixation portion 12, a vacuum port 18 connected to said eye fixation portion, and adjustment arms 20 connected to said eye fixation portion. As shown in FIGS. 1, 2, and 5, eye fixation apparatus 10 can further be provided with annular X-translation and Y-translation guide members 40, 60.

As shown in FIGS. 3, 4, and 5, eye fixation portion 12 is preferably a short toroidal cylinder, with a flat portion on the top 26, and on the bottom provided with an annular convex contact portion 14 which is shaped to conform to the surface of the eye globe and to encircle the cornea. Contact portion 14 is provided with two or more criss-crossing channels 16, exposed on the bottom, which are in communication with the vacuum port 18 for providing vacuum suction to the eye globe conjunctiva attached to the sclera. When placed on the eye, with the contact portion 14 contacting directly upon the eye and encircling the cornea, the criss-crossing channels 16 are upon the eye globe conjunctiva. Vacuum port 18 communicates with is channels 16 such that vacuum pressure exerted at the vacuum port 18 creates vacuum pressure in the criss-crossing channels 16, sucking the eye globe conjunctiva attached to the sclera flush against the contact portion 14. This fixates the eye. The criss-crossing channels 16 work to oppose the suction created by each other, such that the eye glove conjunctiva attached to the sclera, is spread taut between the channels 16, instead of being sucked upon into a particular channel. The criss-crossing channels 16 spread the pressure differential created by the vacuum across the eye globe creating a balanced substantially uniform pressure differential, thus avoiding deformation of the eye globe conjunctiva and sclera in the particular channel. This allows the apparatus to be moved, if an incorrect alignment is made, by shutting off the vacuum pressure, moving the apparatus to the correct alignment, and reasserting the vacuum pressure.

Those skilled in the art will know that the criss-crossing channels 16 can be configured in many different ways to create a substantially uniform pressure differential across the contact portion 14 in relation to the eye globe. Criss-crossing channels can be configured as seen in FIGS. 3 and 4. Criss-crossing channels can be configured as parallel radial grooves with cross channels forming a “train track” like design, as multiple radial grooves with cross channels, as multiple interlocking cross-crossing channels, etc., so long as the configuration allows the pressure differential to be spread substantially uniform across the contact portion in relation to the eye glob.

As shown in FIGS. 1, 2, 3 and 5, vacuum port 18 is a hollow tube extending from and through the eye fixation portion 12 and is in direct communication with the channels 16 which criss-cross the annular convex contact portion 14 of the eye fixation portion 12 such that vacuum pressure applied to said vacuum port 18 exerts vacuum pressure through such criss-crossing channels 16 to pull the eye globe conjunctiva attached to the sclera to the contact portion 14.

As shown in FIGS. 1, 2, and 5, one or more adjustment arms 20 are provided on said eye fixation portion 12 so that the apparatus 10 can be moved without having to directly handle the eye fixation portion 12. Adjustment arms 20 allow the operator to lift the eye fixation apparatus 10 to adjust the fixation to the eyeball.

As shown in FIG. 5 (not shown in FIG. 4), eye fixation apparatus 10 can further be provided with first and second opposing X-translation raised lipped walls 24 on opposite sides of the flat portion 26 on the circular end of the eye fixation portion 12, for slidably interlocking with an X-translation guide member 40.

As shown in FIGS. 1, 2, and 5, eye fixation apparatus 10 can further be provided with an X-translation adjustment apparatus saddle 22. Saddle 22 preferably has four columns extending up from a base, the four columns defining two crossing pathways in which are mounted the X-translation adjustment apparatus 44.

As shown in FIGS. 5 and 6, X-translation guide member 40 is provided with opposing first and second grooves 28 on opposite sides the circular bottom of said member, that match to and interlock with the first and second X-translation raised lipped walls 24. X-translation guide member 40 has a flat portion on the top 56, and is flat on the bottom, between the grooves 28, to rest on the flat portion 26 of the eye fixation portion 12. X-translation guide member grooves 28 fit with X-translation raised lipped walls 24. X-translation guide member slides along flat portion 26 of the eye fixation portion 12. X-translation guide member 40 has first and second Y-translation raised lipped opposing walls 54 on opposite sides of the flat portion 56 on the circular end of the X-translation guide member 40 for slidably interlocking with a Y-translation guide member 60.

As shown in FIGS. 1, 2 and 5, the X-translation guide member 40 is preferably provided with an X-translation adjustment apparatus 44 which mounts on the X-translation adjustment saddle 22 to move the X-translation guide member 40 laterally back and forth in the positive X and negative X direction in relation to the eye fixation portion 12. X-translation adjustment apparatus 44 includes a threaded rod 50, having an end knob 46 on one end, which threads through an adjustment knob 48 that sits in the X-translation adjustment saddle 22, and into threaded aperture 50A of X-translation guide member. Adjustment knob 48 is preferably a circular grooved nut with threads running through center. Adjustment knob 48 sits orthogonal to the X-translation guide member 40 within one of the crossing pathways defined on the X-translation adjustment saddle 22. Threaded rod 50 sits within and runs transversely to the other crossing path of the saddle 22, screwing through the adjustment knob 48, and into the threaded aperture 50A. Turning the adjustment knob 48 moves the X-translation guide member 40 in the positive X and negative X direction.

As shown in FIGS. 1, 2, and 5, X-translation guide member 40 can further be provided with a Y-translation adjustment apparatus saddle 52. Y-translation adjustment apparatus saddle 52 is shaped and operates in the same manner as the X-translation adjustment apparatus saddle 22. Y-translation adjustment apparatus 64 mounts in Y-translation adjustment apparatus saddle 52 in the same manner that the X-translation adjustment apparatus 44 mounts in the X-translation adjustment apparatus saddle 22.

As shown in FIGS. 5 and 6, Y-translation guide member 60 is provided with opposing first and second grooves 28 on the bottom of said member that match to and interlock with the first and second Y-translation raised lipped walls 54 on X-translation guide member. Y-translation guide member 60, between the grooves 28, is flat on the bottom to rest on the flat portion 56 between the Y-translation raised lipped walls 54 on the X-translation guide member 40. Y-translation guide member 60 slides along flat portion 56 of X-translation guide member 40.

Y-translation guide member 60 is provided with a Y-translation adjustment apparatus 64 which mounts to and sits in the Y-translation adjustment saddle 52 to move the Y-translation guide member 60 laterally back and forth in the positive Y and negative Y direction. Y-translation adjustment apparatus 64 includes a threaded rod 70, having an end knob 66 on one end, that threads through an adjustment knob 68 that sits in the Y-translation adjustment saddle 52, and into a threaded aperture 70A in the Y-translation guide member 60. Adjustment knob 68 is preferably a radial circular grooved nut with threads running through center. Turning the adjustment knob 68 moves the Y-translation guide member in the positive Y and negative Y direction.

Those skilled in the art will know that the X-translation guide member 40 and the Y-translation guide member 60 can be configured to translate respectively in any nonparallel lateral direction with respect to each other.

Those skilled in the art will know that in alternative embodiments the Y-translation guide member 60 can easily be connected directly to the eye fixation portion 12, without having an X-translation guide member 40: Those skilled in the art will know that the eye fixation portion 10 can be used without X-translation member 40 or Y-translation member 60.

As shown in FIGS. 1, 2, and 5, a docking screw 72, threaded on one end screws through the Y-translation cutting guide member 60 for tightening the guide member against objects inserted into the: cylindrical space formed by the eye fixation portion 12 and guide members 40, 60. Those skilled in the art will know that docking screw can easily be placed in the eye fixation portion 12, or the X-translation guide member 40.

Apparatus parts can be made of steel, or titanium, or other metals of sufficient strength and sterilizability known to those skilled in the art. Parts can be machined, cast, and etching may be used. Parts can also be made of hard plastic with similar sterilaziblity, tensile strength, and ability to be machined, known to those skilled in the art. Plastic can be machined or injection molded.

In operation in one embodiment, the apparatus 10, held by the adjustment arms 20, is placed upon the surface of the eyeball, to encircle the cornea, with the contact portion 14 directly contacting the eye globe conjunctiva. A vacuum is turned on which creates a vacuum pressure differential through the vacuum port 18 and into the criss-crossing channels 16. If the contact portion 14 placement is not aligned around the cornea, the vacuum is shut off, the apparatus 10 is moved in the globe, using the adjustment arms 20, and the vacuum is reapplied. The user can check for proper centration of the cornea using either direct visualization, placement of a laser docking cone, or by placing a targeting eyepiece into the cylindrical space formed by the first and second annular guide members 40, 60. The apparatus 10 can be used with an applanation lens for surgery. Alternatively, the apparatus 10 can be used for surgery without an applanation lens, where the operation requires fixating the cornea.

Those skilled in the art will recognize that numerous modifications and changes may be made to the preferred embodiment without departing from the scope of the claimed invention. It will, of course, be understood that modifications of the invention, in its various aspects, will be apparent to those skilled in the art, some being apparent only after study, others being matters of routine mechanical, chemical and electronic design. No single feature, function or property of the preferred embodiment is essential. Other embodiments are possible, their specific designs depending upon the particular application. As such, the scope of the invention should not be limited by the particular embodiments herein described but should be defined only by the appended claims and equivalents thereof.

I claim:

1. An eye fixation apparatus, comprising:
an eye fixation portion, wherein the eye fixation portion has an annular convex bottom contact portion, which goes upon the surface of an eyeball and encircles the cornea, and wherein the contact portion is provided with criss-crossing channels; and
a vacuum port connected to said eye fixation portion and in communication with said criss-crossing channels such that vacuum pressure applied to said vacuum port exerts vacuum pressure through such criss-crossing channels to pull the eyeball membrane to the criss-crossing channels.

2. The eye fixation apparatus of claim 1, further comprising adjustment arms connected to said eye fixation portion.

3. An eye fixation apparatus of claims 1 or 2, further comprising a first annular translation guide member adjustably connected to the eye fixation portion, wherein the first translation guide member portion can translate laterally in relation to the eye fixation portion.

4. The apparatus of claims 3, wherein the first translation guide member is provided with a first translation rod and a first adjustment knob for translating the first translation guide member.

5. The apparatus of claims 3, further comprising a docking screw screwed through the first translation guide member for tightening the first translation guide member against objects inserted into the cylindrical space formed by the first annular translation guide member.

6. The apparatus of claims 4, further comprising a docking screw screwed through the first translation guide member for tightening the first translation guide member against objects inserted into the cylindrical space formed by the first annular translation guide member.

7. The apparatus of claims 4, further comprising a second translation guide member adjustably connected to the first translation guide member, wherein the second translation guide member can translate laterally in relation to the first translation guide member in a direction not parallel to the translation of the first translation guide member.

8. The apparatus of claims 7, wherein the second translation guide member is provided with a second translation rod and an adjustment knob for adjusting the second translation guide member.

9. The apparatus of claims 7, further comprising a docking screw screwed through the second translation guide member for tightening the second translation guide member against objects inserted into the cylindrical space formed by the annular second translation guide member.

10. The apparatus of claims 8, further comprising a docking screw screwed through the second translation guide member for tightening the second translation guide member against objects inserted into the cylindrical space formed by the annular second translation guide member.

11. A method of fixating an eye cornea for surgery, comprising:
placing an eye fixation apparatus upon the eye globe conjunctiva around the cornea, wherein the eye fixation apparatus comprises an eye fixation portion with an annular convex bottom contact portion provided with criss-crossing channels, and a vacuum port connected to said eye fixation portion and in communication with said criss-crossing channels such that vacuum pressure applied to said vacuum port exerts vacuum pressure through such criss-crossing channels to pull the eyeball membrane to the criss-crossing channels; and
applying vacuum pressure to said vacuum port creating a pressure differential through said criss-crossing channels in relation to said conjunctiva, adhering said conjunctiva to said contact portion.

12. The method of claims 11, further comprising:
checking to see said eye fixation apparatus is centered around the cornea; and
shutting off the vacuum pressure if said eye fixation apparatus is not centered around the cornea, recentering said eye fixation apparatus, and reapplying said vacuum pressure.

13. The method of claims 11 or 12, wherein the eye fixation apparatus is further provided with adjustment arms connected to said eye fixation portion.

14. The method of claims 11 or 12, wherein the eye fixation apparatus further comprising a first annular translation guide member adjustably connected to the eye fixation portion, wherein the first translation guide member portion can translate laterally in relation to the eye fixation portion.

15 The method of claims 14, wherein the first translation guide member is provided with a first translation rod and a first adjustment knob for translating the first translation guide member.

16 The method of claims 13, wherein the eye fixation apparatus is further provided with a docking screw screwed through the first translation guide member for tightening the first translation guide member against objects inserted into the cylindrical space formed by the first annular translation guide member.

17. The method of claims 14, wherein the eye fixation apparatus is further provided with a docking screw screwed through the first translation guide member for tightening the first translation guide member against objects inserted into the cylindrical space formed by the first annular translation guide member.

18. The method of claims 14, wherein the eye fixation apparatus is further provided with a second translation guide member adjustably connected to the first translation guide member, wherein the second translation guide member can translate laterally in relation to the first translation guide member in a direction not parallel to the translation of the first translation guide member.

19. The method of claims 18, wherein the second translation guide member is provided with a second translation rod and an adjustment knob for adjusting the second translation guide member.

20. The method of claims 18, wherein the eye fixation apparatus is further provided with a docking screw screwed through the second translation guide member for tightening the second translation guide member against objects inserted into the cylindrical space formed by the annular second translation guide member.

21. The method of claims 19, wherein the eye fixation apparatus is further provided with a docking screw screwed through the second translation guide member for tightening the second translation guide member against objects inserted into the cylindrical space formed by the annular second translation guide member.
